(19) United States
(12) Patent Application Publication
(10) Pub. No.: US 2017/0027767 A1
(43) Pub. Date: Feb. 2, 2017

(54) DEVICE FOR TREATING THE FEMALE UROGENITAL TRACT

(71) Applicants: Corinna Hengsberger, Schopfheim (DE); Otto von Stetten, Aachen (DE); TECURO AG, Luzern (CH)

(72) Inventors: Corinna Hengsberger, Schopfheim (DE); Otto von Stetten, Aachen (DE)

(73) Assignee: TECURO AG, Luzern (CH)

(21) Appl. No.: 15/302,492

(22) PCT Filed: Apr. 6, 2015
(86) PCT No.: PCT/DE2015/000184

§ 371 Date: Oct. 7, 2016

(30) Foreign Application Priority Data
Apr. 9, 2014 (DE) 10 2014 005 282.6

(51) Int. Cl. A61F 13/472; A61F 13/47

(52) U.S. Cl. CPC A61F 13/472; A61F 13/47227; A61F 13/4704; A61F 2013/8414

(57) ABSTRACT

The invention relates to a device for treating the female urogenital tract and is characterized in that the device comprises a covering device (1) for the urogenital region at least in the region of the vulva, the vagina and the urethra; and in that the covering device is treated substantially fully with an antimicrobial and/or antiseptic substance preventing the migration of bacteria into the urogenital tract.

The invention relates to a device for treating and cleaning the female urogenital tract.

Kidney and urinary passage infections are frequent infectious diseases which women tend to contract substantially more frequently than men because of anatomical conditions. Approximately 10% of women suffer from relapsing infections with three or more episodes per year. These infections are mainly caused by E. coli bacteria, but also by Klebsiella, Proteus, or Staphylococcus being originated in their own intestine. From the anus they migrate into the urethra and eventually the bladder through the skin, and from there into the renal pelvis. Even after recovery from an infection, niches of surviving bacteria may form at the entrance of the urethra, which may lead to a relapse.

Antibiotics are systematically used for therapy purposes, however, they become ineffective due to the resistance of the bacteria resulting from the frequency of application required, and they also produce undesirable side effects.

The currently popular phytotherapy, mainly based on cowberries or cranberry, delivered variable results in studies and has not been validated so far.

Patent application US 2002/0115976 describes a device for treating the female urogenital tract in the form of a sanitary towel, which covers the female anus area, the perineum and the genital area. A mechanical “barrier” is provided in the central perineum area, which extends transversely on the sanitary towel in the perineum area between the anus and the genital area. This “barrier” may present anti-microbial substances, so that, as is stated therein, no micro-organisms or bacteria may migrate from the anus to the genital area. Nevertheless, micro-organisms or bacteria cannot be blocked by a barrier, even if this barrier contains anti-microbial substances, since these organisms are able to find all possible ways and cannot be retained by any mechanical barrier.

The object of the invention is to develop a novel therapy approach for treating the female urogenital tract and to specify a device which enables to reliably avoid infections in the urogenital tract and to effectively treat existing infections.

This object is solved by the features of claim 1.

According to the invention, instead of a systemic treatment based on medicines, particularly antibiotics, local treatment of the urogenital tract by anti-microbial or antiseptically active substances is used, with which a covering of the urogenital tract is prepared in a substantially complete way. Through this preparation, all bacteria are completely eliminated.

The covering covers preferably at least the vulva, the vagina and the female urethra. However, it can also cover the anus area, namely with a first cover layer, which comes into contact with the skin, an intermediate layer, which may optionally exhibit absorbing features, and a second cover layer, which closes the device or the covering on the other side, the layer facing the skin being coated with a layer of an anti-microbial or antiseptically active substance or mixture.

The two outer layers may be formed of the same or a different material which is compatible with the skin. For this purpose, fabrics and non-woven materials can be taken into consideration, for example, of cotton, polypropylene, polyester, polyethylene, polyamide, nylon, etc. The surface of the layers, particularly of the layer facing the skin, may be optionally designed in a structured way. The intermediate layer contributes to the comfort of the device when being worn and may also optionally exhibit absorbing features. In this case, wadding, polyurethane foam, polyethylene, polypropylene fabrics or non-woven materials may be taken into consideration, which can also be doped with absorbent materials, such as superabsorbent polymers.

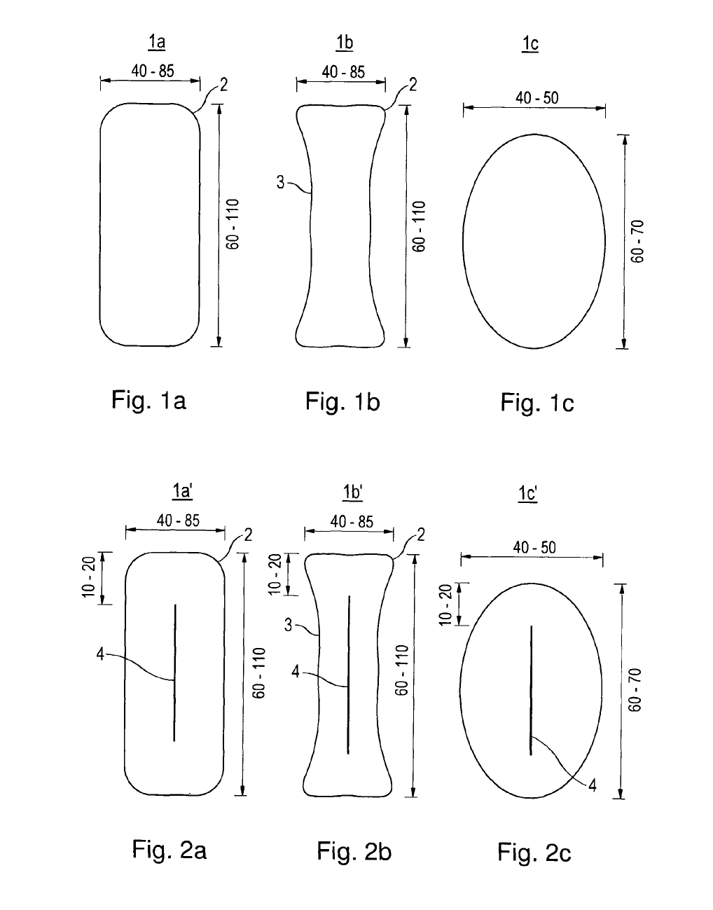

The shape of the device corresponds to the conditions of the anatomy in the urogenital region. Thus, it is elongated and rounded at the ends, similar to the shape of known sanitary towels. FIG. 1 shows examples thereof. For the anti-microbial effectiveness, a close contact of the device with the skin/mucous membrane in the urogenital region is crucial.

In an embodiment according to the invention, in order to enhance this effectiveness, the skin-facing side of the device can present a topstitch which lightens the folding and so the anatomical adapting, represented by way of example in Drawing 2.

In a further configuration of the invention, the shape of the device is chosen so that this can be placed and worn in an interlabial way, as shown by way of example in Drawings 1c and 2c.

In a particularly preferred configuration, the side turned towards the skin is coated, either in stripes or punctually with muco or bioadhesive materials, which fix the device close to the skin or the mucous membrane. In this aspect, carbomers, poloxamers, polyacrylates and derivatives, chitosan and chitosan derivatives, alginate polyethylene glycol acrylates, thiomers (e.g. polycarbophil-cysteine, etc.,) lectins, etc., and mixtures of the aforementioned materials, for example, may be taken into consideration.

Since the device remains for a long time at the selected location, the layer turned towards the skin may be further provided with substances or mixtures which increase the skin humidity. For this purpose, glycerol, hyaluronic acid, glycols, lactates, urea, etc., for example, are suitable. Thereby a high dehydration of the skin and possible frictions derived from the use of the device are prevented, and the use is more comfortably conceived.

In order to avoid problems regarding the development of resistance in bacteria, according to the invention no antibiotics, but skin-friendly antiseptic and anti-microbial substances are used, from which no development of resistance is known. The substances are selected from the known active agents, such as surfactants, quaternary ammonium compounds, biguanides, organic acids, metal ions, such as copper, zinc, silver, gold, ruthenium, rhodium, palladium, etc., iodine compounds, aldehydes, etc. and mixtures thereof. Three of the multiply conceivable possibilities are shown below by way of example.

In an embodiment of the invention, a mixture of a biguanide (polyhexamethylene biguanide), a quaternary ammonium compound (benzalkonium chloride), and an amphoteric surfactant (undecylenic amidopropyl betaine) is applied on the skin-facing side of the device.

In a further embodiment, the device is coated with a mixture of gold, silver and palladium, as it is commercially available under the designation Bactiguard. A particularly preferred embodiment uses mixtures of vitamin C (ascorbic acid), organic acids (tartaric acid, citric acid), cuprous salts (copper chloride) and a surfactant (lauryl sulphate) as described in EP 2190398.

The device is manufactured following the conventional methods for manufacturing panty liners, eye pads, and the like. In these methods, the three layers of the unworked piece are joined to one another by means of stamping, sealing, adhesives or using microwaves or ultrasounds. In a second step, the first cover layer is coated with the anti-microbial active agent or the mixture. For this purpose, the unworked piece can be immersed in an active agent solution, be sprayed with this or pressed against it, or be manufactured by similar methods.

In a further method, the first cover layer is doped with the active agent or active agent mixture in a first step. Optionally, bio or mucoadhesive substances are also applied. This “active” layer is connected according to the usual methods with the other two layers in a second step.

For the purpose of microbially cleaning the urogenital region, the cover of the device according to the invention is folded in the middle and is placed so that it covers the skin and the mucous membrane of the urogenital region. Thus, an anti-microbial barrier is formed between the anus and the urogenital region, which prevents bacteria from migrating into the urethra and eliminates them.

In a particularly preferred embodiment, the device is placed in an interlabial way, i.e. between the labia, which enhances the effectiveness due to the close contact.

The following example illustrates the invention in more detail without restricting its scope.

A pre-assembled device, corresponding for instance Drawing 1c in the form and whose cover layer consists of a mixed material of polyethylene and polypropylene was impregnated with approximately 0.4 g of an aqueous solution containing lauryn sulphate, ascorbic acid, citric acid, tartaric acid, copper chloride, and was air-dried for 6 hours. The anti-microbial activity was analysed in a practical test with artificial human skin and Escherichia coli (E. coli).

Efficacy Test of Antibacterial Textiles in a Simulated Use on Human Skin Flora

The practical examination is based on a standardised, artificial/technical skin (HUMskin), colonised with Escherichia coli ATCC 8739. HUMskin is a standardised technical skin-replacing material based on a biopolymer which simulates the topographic and physiological features of healthy skin.

The effect of the testing material can be represented by a SEM-image of the HUMskin surface with germ colonisation.

The effect of the testing material on the testing germ is quantitatively analysed in a simulated use on the artificial skin during a contact time of 6 hours (6 h) and evaluated against an internal control (Standard PES) or a reference material.

For this purpose, the HUMskin (3 cm²) is contaminated at the 0 h moment with a defined number of E. coli germs (at the 0 h moment) and is covered with the control/reference material or the sample and incubated for 6 hours at 36±1° C. (each with a 3-fold approach). Once the incubation is finished, the remaining number of germs on the HUMskin is determined by the control/reference and the sample (absolute CFU at the 6 h″ moment, each with a 3-fold approach).

The antibacterial activity is calculated according to the formula

A=(log10C6h−log 10T6h)

calculated with C=control material and T=sample material.

The following results were obtained:

| | Absolute | | Log CFU | Log CFU |
|---|---|---|---|---|
| Sample | CFU 6 h | Log CFU | vs. Control | vs. Reference |
| Control | 1.25 × 10⁶ | 6.10 | | |
| Reference B | 1.96 × 10³ | 6.29 | −0.20 | |
| B + Active agent | <20 | ≦1.28 | ≦4.82 | ≦5.01 |

This means that bacteria were reduced by more than 99.999%. The device is effective to a great extent and solves the object of the invention.

The figures show schematically devices or covers for the urogenital tract of a woman; in these:

FIG. 1a) shows a first exemplary embodiment of an elongated, approximately rectangular, cover;

FIG. 1b) shows a second exemplary embodiment of an also elongated cover;

FIG. 1c) shows a cover in an oval form;

FIGS. 2a), 2b) and 2c) show top views of covers according to the invention corresponding to FIGS. 1a), 1b) and 1c), each having an intermediate long stitching.

Dimensions are given in millimetres in all figures.

Thus, a cover la according to FIG. 1a) is, for instance, rectangular and presents a length of approx. 60 to 110 millimetres (mm). The narrow side has a width of 40 to 85 mm, the covering being rounded at its four corners 2. The dimensions may change depending on a woman's height. The cover extends over the vulva, the vagina and the female urethra when it is being used. The length of the cover may be larger, particularly in the case that covering the anus is also desired; in this case, it can be of about 200 mm.

The cover 1b according to FIG. 1b) presents longitudinal tapering ends 3 on the longitudinal sides, beginning from the rounded corners 2, in order to ensure an anatomically comfortable fit. Dimensions are otherwise similar to those in FIG. 1a).

The cover 1c (according to FIG. 1c) is oval, with a length of 60 to 70 mm and a width of 40 to 50 mm.

Covers 1a′, 1b′ and 1c′ correspond in their dimensions to covers 1a, 1b and 1c respectively, wherein respectively an intermediate stitching is provided in the longitudinal direction of the cover, for example, a topstitch 4 or the like, which begins respectively at about 10 to 20 mm from the upper or the lower edge of the cover. The cover may be hereby folded in this area in order to reach a close contact, also an interlabial contact at the vulva of the woman.

1. Device for treating thc femalo an urogenital tract of a female comprising:
a cover for an urogenital area at least in an area of a vulva, a vagina and an urethra of the female,
wherein the cover comprises an anti-microbial and/or antiseptically active substance against migration of bacteria into the urogenital tract.

2. The device according to claim 1, wherein the cover also covers the female's anus.

3. The device according to claim 1, wherein the cover comprises at least one layers.

4. The device according to claim 3, wherein the cover comprises substantially three layers, namely
a first cover layer covering a first side of the device which comes into contact with skin of the female,
an intermediate layer, which optionally has absorbing features, and
a second cover layer, which closes the device on a second side of the device,
the first cover layer comprising or being coated with the anti-microbial, the antiseptically active substance or a mixture thereof.

5. The device according to claim 1, wherein the cover has an elongated shape of a sanitary towel, which has ends that are rounded.

6. The device according to claim 4, wherein the first cover layer comprises a topstitch in its middle adapted to simplify folding of the cover.

7. The device according to claim 1, wherein a shape of the device is configured so that the device can be placed and worn in an interlabially way.

8. The device according to claim 4, wherein the intermediate layer comprises a material which exhibits the absorbing features.

9. The device according to claim 4, wherein the first and optionally the second cover layer comprise a skin-compatible fabric.

10. The device according to claim 4, wherein the first cover layer is coated with bio- or mucoadhesive substances, either in stripes or in points, for a better fixation on the skin of the female.

11. The device according to claim 4, wherein the first cover layer is additionally coated with skin moisture-increasing substances.

12. The device according to claim 4, wherein the active substance or the mixture is are surfactants, quaternary ammonium compounds, biguanides, organic acids, metal ions, including copper zinc, silver, ruthenium, rhodium, iodine compounds, aldehydes, and mixtures thereof.

13. The device according to claim 4, wherein the mixture is applied onto the device that has been pre-assembled by immersing, spraying, or pressing.

14. Method for manufacturing a device according to claim 4, comprising:
coating the first cover layer with the active substance or the mixture and subsequently joining the first cover layer with the intermediate layer and the second cover layer.

15. Method for microbially cleaning an urogenital region of a female in need thereof, comprising interlabially placing the device according to claim 1 to clean the urogenital region of the female.

16. The device according to claim 3, wherein the cover comprises one or more textile layers.

17. The device according to claim 3, wherein the cover comprises more than one layer.

18. The device according to claim 8, wherein the material which exhibits absorbing features is wadding, polyurethane foam or superabsorbent polymers.

19. The device according to claim 9, wherein the skin-compatible fabric is cotton, polypropylene, polyester or polyethylene.

20. The device according to claim 11, wherein the skin moisture-increasing substance is glycerol, hyaluronic acid, glycols, or urea
